(19) United States
(12) Patent Application Publication
(10) Pub. No.: US 2025/0000625 A1
(43) Pub. Date: Jan. 2, 2025

(54) DISK MANAGER FOR DENTAL BLANKS

(71) Applicant: Ivoclar Vivadent AG, Schaan (LI)

(72) Inventors: Tarkan CETIN, Feldkirch (AT); Sebastian GASSNER, Triesenberg (LI); Andreas GANTIOLER, Bludenz (AT); Martin BERTAGNOLLI, Naturns (IT)

(21) Appl. No.: 18/756,317

(22) Filed: Jun. 27, 2024

(30) Foreign Application Priority Data
Jun. 29, 2023 (EP) 23182292.5

(51) Int. Cl. A61C 13/00; A61C 19/10

(52) U.S. Cl. CPC A61C 13/0022; A61C 19/10

(57) ABSTRACT

A disk manager (100) for managing dental blanks (101-1, . . . , 101-n), comprising several receiving compartments (103-1, . . . , 103-n) for receiving one dental blank (101-1, . . . , 101-n) each; and a display device (105-1, . . . , 105-n) for displaying the receiving compartment (103-1, . . . , 103-n) in which a dental blank (101-1, . . . , 101-n) is received.

### CROSS-REFERENCE TO RELATED APPLICATIONS

This application claims priority to European Patent Application No. 23182292.5 filed on Jun. 29, 2023, the disclosure of which is incorporated herein by reference in its entirety.

### TECHNICAL FIELD

The present invention relates to a disk manager for managing dental blanks, a module for a disk manager comprising at least one receiving compartment for receiving a dental blank and a method for managing dental blanks.

### BACKGROUND

Finding disk-shaped or block-shaped dental blanks is often associated with a search effort in the dental laboratory workflow. Once the correct material for a dental restoration has been selected, it is possible to choose between different dental blanks of the same type from which dental restorations have already been fabricated. The more dental blanks are available, the longer it takes to find the correct blank.

If the dental blank with the least amount of remaining material is not selected or new dental blanks are used before available remaining material is used up, the stock level is unnecessarily increased. In addition to selecting and searching for the dental blanks, time is required to insert and remove the dental blanks from the disk holders.

When nesting or activating new dental blanks, they are first nested on a computer before being loaded into the manufacturing machine. If the computer and the manufacturing machine are physically separated from each other, this results in a further loss of time.

Stacking or storing the blanks on tables, in drawers or on shelves can lead to breakage at the edges of the blanks due to frequent handling. Once a dental blank is completely worn out, it is disposed of and a new one is ordered. The remaining stock of the respective material in the storehouse also has an influence on the ordering time and an order recommendation.

US20200337817 is directed to a mill blank inventory monitoring and dispensing system and is hereby incorporated by reference.

### SUMMARY

It is the technical task of the present invention to reduce the handling effort of dental blanks.

This task is solved by objects according to the independent claims. Technically advantageous embodiments are the subject-matter of the dependent claims, the description and the drawings.

According to a first aspect, the technical task is solved by a disk manager for managing dental blanks, comprising several receiving compartments for receiving one dental blank each; and a display device for displaying the receiving compartment in which a dental blank is received. The blank can be block-shaped or disk-shaped. The disk manager can reduce the search effort for dental blanks after an order has been sent by the CAM software or the manufacturing machine, as only the blank that is indicated by the display device is picked, for example where a light-emitting diode is on (pick-by-light). Contact with the blank is only made when the blank is actually needed. Loose stacking of the blanks can be dispensed with.

In a technically advantageous embodiment of the disk manager, the disk manager comprises a storage device for storing the properties of the dental blank in the respective receiving compartment. The storage device is, for example, a digital data memory for digitally storing information about the properties of the blank, such as a RAM memory or a hard disk. The data can also be stored online or in a network. The disk manager can also comprise a read and write function via RFID readers. The read data can then be further processed in a network. The properties of the dental blank can comprise nesting data, in which the size and shape of the milled-out areas that have previously been milled out of the dental blank are specified. The nesting data can be used to determine whether further dental restorations can be milled out of the respective blank.

This provides the technical advantage, for example, that an electronic storage of dental blanks can be performed.

In a further technically advantageous embodiment of the disk manager, the properties comprise available remaining material, a color, an identification number, a size and/or a manufacturing material of the received blank. This information can be stored, managed and evaluated in CAM software. This provides the technical advantage, for example, that the blanks can be managed on the basis of particularly suitable properties.

In a further technically advantageous embodiment of the disk manager, the disk manager can be composed of several modules with at least one receiving compartment. This provides the technical advantage, for example, that the disk manager can be adapted to spatial conditions.

In a further technically advantageous embodiment of the disk manager, the disk manager comprises an input interface for inputting properties of a dental blank. The input interface is used to read in the blank. The disk manager can also comprise an input interface for communicating with CAM software or a manufacturing machine. This provides the technical advantage, for example, that data on blanks with desired properties can be transmitted to the disk manager.

In a further technically advantageous embodiment of the disk manager, the receiving compartments comprise an inclination or a foam insert for stable positioning of the dental blank. This provides the technical advantage, for example, of preventing the blank from unintentionally rolling out.

In a further technically advantageous embodiment of the disk manager, the disk manager comprises a scanning device for scanning a dental blank. This provides the technical advantage, for example, that data on properties of the blank can be easily determined. The data can be compared or exchanged via a network.

In a further technically advantageous embodiment of the disk manager, the scanning device comprises an optical camera, an RFID reader, a bar code reader and/or a QR code reader. This provides the technical advantage, for example, that the blank can be scanned easily and quickly.

In a further technically advantageous embodiment of the disk manager, an antenna or a sensor for detecting the received dental blank is assigned to each receiving compartment. The antenna or the sensor detects whether a dental blank is received in the receiving compartment. The sensor can be a mechanical button, a pressure sensor or an ultrasonic sensor. This provides the technical advantage, for example, that a current actual occupancy of the receiving compartments is known.

In a further technically advantageous embodiment of the disk manager, a display device is assigned to each receiving compartment. This provides the technical advantage, for example, that the receiving compartment assigned to the blank can be recognized easily and quickly.

In a further technically advantageous embodiment of the disk manager, the display device comprises a light-emitting diode, a liquid crystal display or an electronic paper. The light-emitting diode can be color-tunable. This provides the technical advantage, for example, that the receiving compartment assigned to the blank can be clearly identified.

According to a second aspect, the technical task is solved by a module for a disk manager with at least one receiving compartment for receiving a dental blank. The module achieves the same technical advantages as the disk manager according to the first aspect.

In a technically advantageous embodiment of the module, the module comprises a display device for displaying the receiving compartment. The display device can also be formed on the blank. This provides the technical advantage, for example, that the receiving compartment assigned to a blank can be displayed on the module.

In a further technically advantageous embodiment of the module, the module can be coupled horizontally or vertically. This provides the technical advantage, for example, that the disk manager can be set up in a spatially flexible manner. This is also generally conceivable for a disk manager. In general, the modules can also be arranged spatially separately. Not only horizontally and vertically, but also spatially separated.

According to a third aspect, the technical task is solved by a method for managing dental blanks, comprising the steps of receiving a dental blank in one of several receiving compartments; and displaying the receiving compartment in which a dental blank with predetermined properties is received. The method achieves the same technical advantages as the disk manager according to the first aspect.

In a technically advantageous embodiment of the method, it is detected whether a dental blank is inserted in one of the receiving compartments. This provides the technical advantage, for example, that an occupancy of the receiving compartments can be determined.

In a further technically advantageous embodiment of the method, the remaining quantity of a dental blank is detected. This provides the technical advantage, for example, that a stock level can be managed.

In a further technically advantageous embodiment of the method, a dental blank is automatically reordered if the remaining quantity falls below a predetermined value. This provides the technical advantage, for example, that a sufficient number of each dental blank is available.

In a further technically advantageous embodiment of the method, a stock level of dental blanks is managed. This provides the technical advantage, for example, that the dental blanks can be managed in such a way that sufficient quantities are always available.

### BRIEF DESCRIPTION OF THE DRAWINGS

Exemplary embodiments of the invention are shown in the drawings and are described in more detail below, in which:

FIG. 1 shows a schematic view of a disk manager;

FIG. 2 shows a schematic view of a disk manager built modularly; and

FIG. 3 shows a block diagram for managing dental blanks.

### DETAILED DESCRIPTION

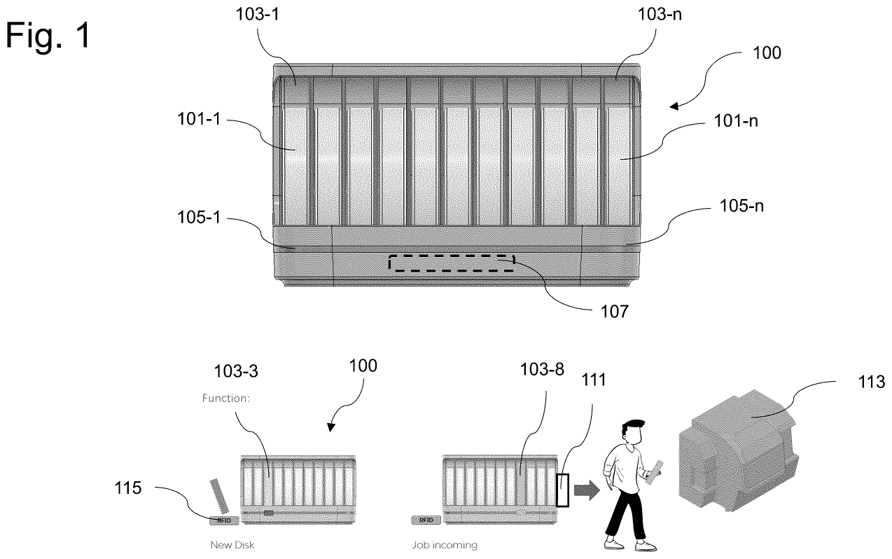

FIG. 1 shows a schematic view of a disk manager 100. The disk manager 100 forms a structured storage system and comprises several receiving compartments 103-1, . . . , 103-n for receiving a dental blank 101-1, . . . , 101-n each. Furthermore, the disk manager 100 comprises a display device 105-1, . . . , 105-n for displaying the receiving compartment 103-1, . . . , 103-n in which a specific dental blank 101-1, . . . , 101-n is received.

The blanks 101-1, . . . , 101-n can be stored individually in the receiving compartments 103-1, . . . , 103-n. The shape of the receiving compartments 103-1, . . . , 103-n is adapted to the blanks 101-1, . . . , 101-n. For example, the rear wall adapts to the radius of the blank. With an inclination, the blanks 101-1, . . . , 101-n roll gently into the receiving compartments 103-1, . . . , 103-n and do not roll out of the disk manager 100. This can be further prevented by an additional edge or lip above the display device 105-1, . . . , 105-n. The receiving compartments 103-1, . . . , 103-n can be equipped with foam. This can prevent breakage of the blanks 101-1, . . . , 101-n. Each receiving compartment 103-1, . . . , 103-n can comprise its own antenna. An antenna multiplexer thus recognizes which receiving compartment 103-1, . . . , 103-n is occupied.

The disk manager 100 comprises a digital storage device 107 for storing data on the properties of the dental blank 101-1, . . . , 101-n, which is received in the respective receiving compartment 103-1, . . . , 103-n. It is also possible to store data on the properties of the dental blank 101-1, . . . , 101-n in the network. The properties relate, for example, to available remaining material, a color, an identification number, a size and/or a manufacturing material of the received blank. The disk manager 100 also comprises a processor (CPU) for evaluating the stored data.

ROM, RAM and HD are digital storage devices 107 for storing data and computer-executable instructions that can be executed by the processor or compiled or interpreted to be executed by the processor. Suitable computer-executable instructions may be on a computer-readable medium (e.g., ROM, RAM, and/or HD), hardware circuitry, or the like, or any combination thereof.

The storage device 107 may comprise any type of data storage medium that can be read by a processor. Examples of computer-readable storage media may comprise, but are not limited to, volatile and non-volatile computer memory and storage devices, such as random-access memory, read-only memory, hard disk drives, direct access storage device arrays, flash memory drives, optical data storage devices, read-only compact disk memory, and other suitable computer memory and data storage devices. The storage device 107 may also be formed by radio frequency identification tags (RFID tags) on the respective blanks.

The disk manager 100 may comprise one or more processors coupled to one or more user input/output (I/O) devices and storage devices. Examples of I/O devices may comprise, but are not limited to, keyboards, displays, monitors, touchscreens, printers, electronic pointing devices such as mice, trackballs, pens, touchpads, or the like.

The disk manager 100 can be coupled with a display, an information device and various peripheral devices such as printers, plotters, speakers. The disk manager 100 can also be coupled to external computers or other devices via a network interface, wireless transceiver or other means that is coupled to a network, such as a local area network (LAN), wide area network (WAN) or the Internet.

An input interface 111 can be used to feed data about the properties of a searched-for blank 101-1, . . . , 101-n, for example via CAM software. The disk manager 100 compares this data with the stored data of the received blanks 101-1, . . . , 101-n and uses the display device 105-1, . . . , 105-n to display the receiving compartment 103-1, . . . , 103-n in which the blank 101-1, . . . , 101-n with the desired properties is received. This allows the blanks to be stored chaotically, as the algorithm always finds an existing blank 101-1, . . . , 101-n.

Sorting of the blanks 101-1, . . . , 101-n can also be performed with structure based on sorting algorithms or machine-learning algorithms in a digital library. The disk manager 100 can also perform a suggestion for an optimal or waste-optimized blank 101-1, . . . , 101-n. Filtering and sorting of the blanks 101-1, . . . , 101-n can be performed in the CAM software.

First, for example, a new milling order is received from a manufacturing machine. If the dental blank 101-8 with the desired material is stored in the disk manager 100, the respective receiving compartment 103-8 lights up orange. When the blank 101-8 is removed from the disk manager 100, placed in the manufacturing machine 113 and recognized by the latter, the respective receiving compartment 103-8 on the disk manager 100 goes out.

If a new blank 101-3 is inserted in the meantime, it can be stored on the disk manager 100 via an RFID reader as a scanning device 115 in a CAM library, a central database or on a cloud server and stored in a receiving compartment 103-3 lit up green.

Once the job is complete, the blank 101-8 is removed from the manufacturing machine 113 again, also scanned at the RFID reader on the disk manager 100 and then stored again in the receiving compartment 103-8 lit up green. The blank 101-8 can also be stored in any free receiving compartment 103 or in a receiving compartment 103 that is selected using a sorting algorithm. This can also be done randomly, as the blanks are fed out of a manufacturing machine at different times depending on the milling job.

The blank 101-1, . . . , 101-n can also be scanned during the insertion of the blank 101-1, . . . , 101-n into the receiving compartment 103-1, . . . , 103-n. For this purpose, a receiving compartment 103-1, . . . , 103-n with an integrated scanner (scan gate) can be used, through which the blank 101-1, . . . , 101-n is scanned on its way into the receiving compartment 103-1, . . . , 103-n. A scanning device 115, which is coupled to the disk manager 100, may be movably arranged on a rail. The scanning device 115 can also be formed by a barcode or QR code reader. The barcode or QR code can be arranged circumferentially around the blank 101-1, . . . , 101-n. When the blank 101-1, . . . , 101-n is scanned, an automatic reorder can be performed. A delivery bill can be transferred to the CAM database. In addition, digital stock management or an analysis and evaluation of nesting data can be carried out.

The nesting data indicates at which point of the dental blank 101-1, . . . , 101-n dental restorations have already been milled out and how much material has been used.

The nesting data can be used to determine at which point and in which size further dental restorations can be milled out of the blank 101-1, . . . , 101-n. This nesting data can be transmitted to a storage cabinet by exchanging information.

The disk manager 100 can also comprise a printer for labeling tags. The creation of a digital ID is independent of the labeling. The labeling can be arranged on the disk holder, the blank or labeling flags. Color codes, barcodes or QR codes can be used for this purpose.

An input interface 111 is used to input properties of a dental blank 101-1, . . . , 101-n. The input interface may, for example, be formed by a data link with a CAM software or by a user interface through which a user can manually enter this data, such as a touch-sensitive screen. The input interface 111 can also be formed by an optical scanner, such as a bar code scanner or a QR code scanner.

The blank 101-1, . . . , 101-n can be scanned and stored on a trolley or holder (2-axis handling). For this purpose, RFID tags can be distributed around the blank 101-1, . . . , 101-n so that it can be read from all sides. The electronic input interface 111 can also be configured in such a way that it retrieves the data stored on the RFID tag of the blank 101-1, . . . , 101-n.

Camera monitoring of the sectional image of the blank 101-1, . . . , 101-n from above enables a comparison with the CAM database and a creation of a sectional image library. The blank 101-1, . . . , 101-n can be read in directly at the manufacturing machine 113 or at the disk manager 100.

The disk manager 100 generates a barcode that is synchronized with the manufacturing machine 113. The barcode reading and printing station can be coupled directly with the disk manager 100. Analogous to the disk manager 100, a block manager can also be designed, which receives the blocks in a matrix design.

The disk manager 100 saves time in the production workflow and optimizes a dental laboratory's stock levels. In addition, space is saved and a clearer disk portfolio is realized. In addition, loss and theft monitoring of the blanks 101-1, . . . , 101-n by the disk manager 100 is possible. For this purpose, the user can be identified by the disk manager 100, for example by means of a lock or personal login.

The most waste-optimized blank 101-1, . . . , 101-n can be prioritized on the disk manager 100 in order to make optimum use of the stock. The disk manager 100 enables a more compact storage of the blanks 101-1, . . . , 101-n compared to a drawer.

The disk manager 100 has a portable form factor with a power supply via a power supply unit, the manufacturing machine 113, a computer (USB) or a battery. The disk manager 100 can also be seen as an external disk changer with integrated order coordination and stock management. The disk manager 100 can be designed as a stand-alone disk manager as a plug-in for a CAM software or for a manufacturing machine.

Connecting the disk manager 100 to a cloud can prevent or enable multiple manufacturing machines 113 from accessing the same blank 101-1, . . . , 101-n. This can prevent access conflicts. In addition, the disk manager 100 can be located next to the manufacturing machine 113 and shorten the transport path from nesting on the computer to the manufacturing machine 113. New blanks 101-1, . . . , 101-n that are still in the packaging can be scanned directly into the CAM software on the disk manager 100. The data is recorded and evaluated using intelligent algorithms so that the most frequently used materials are determined and the optimum receiving compartment 103-1, . . . , 103-n for the respective blank 101-1, . . . , 101-n is determined.

The blank 101-1, . . . , 101-n can also be inserted into any receiving compartment 103-1, . . . , 103-n and the disk manager 100 recognizes by means of a difference adjustment in which receiving compartment 103-1, . . . , 103-n the blank 101-1, . . . , 101-n has been inserted, for example by means of an antenna in each compartment.

Communication about the properties of the blanks 101-1, . . . , 101-n can be wireless or wired between the disk manager 100 and a dental manufacturing machine 113. The blanks 101-1, . . . , 101-n are not only initialized by the disk manager 100 on the basis of the material, but also with the current milling profile, for example how much space is still available for dental restorations on the blank 101-1, . . . , 101-n.

For this purpose, the CAM software and the disk manager 100 communicate and synchronize with each other, i.e. the information about used blanks 101-1, . . . , 101-n is stored and continued until they are disposed of. A comparison can be made between the real blank 101-1, . . . , 101-n and a digital twin in the database (CAM library).

For managing and optimally sorting blanks, the disk manager 100 may be connected to a storehouse and interact with the manufacturing machine 113 and the CAM software. The user receives the optimal blank 101-1, . . . , 101-n for the respective job at the manufacturing machine 113.

Decoupled from the manufacturing machine 113, communication can also only take place between a CAM software and the disk manager 100. This makes it possible to connect the disk manager 100 to different manufacturing machines 113 and to store external blanks (open system).

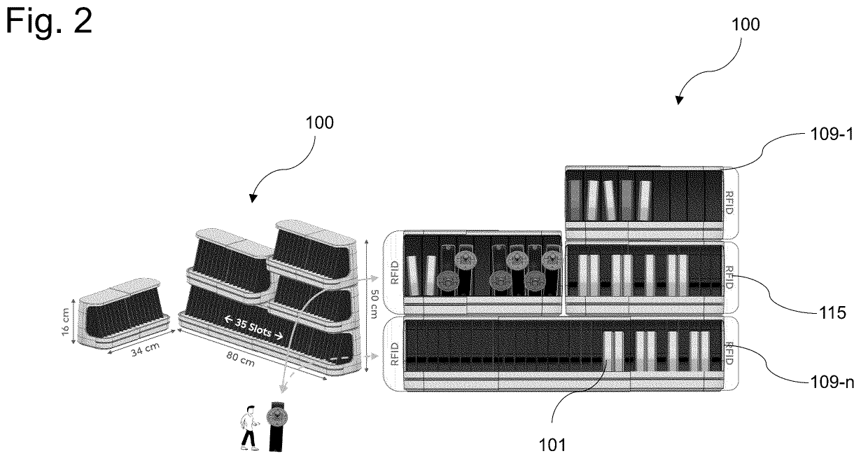

FIG. 2 shows a schematic view of a modularly built disk manager 100. The disk manager 100 comprises several modules 109 with receiving compartments 103-1, . . . , 103-n, which can be assembled horizontally and/or vertically. This design is more space-saving than drawers and is scalable in length and height (C-shape design). It is also possible to stack several disk managers 100 of different sizes. The number of clamping and unclamping operations is reduced for a disk manager 100 with wider receiving compartments 103-1, . . . , 103-n.

The disk manager 100 can be arranged on a lower or upper cabinet on the manufacturing machine at a convenient operating height, so that the blanks 101-1, . . . , 101-n are close to the manufacturing machine. Several disk managers 100 can be divided among several manufacturing machines 113. The disk manager 100 can be built into the manufacturing machine 113, for example in a side wall in which an arm grips the blank 101-1, . . . , 101-n or a magazine is mounted so that it can be moved vertically or horizontally. This can be used to move to a transfer position in order to remove the blank 101-1, . . . , 101-n with a robot arm.

The adaptive design of the disk manager 100 also makes it possible to store disk holders with inserted blanks in addition to the blanks 101-1, . . . , 101-n as such. The disk manager 100 or a module 109 can also be specially configured to receive disk holders with inserted blanks 101-1, . . . , 101-n. At this, different modules can also be combined with each other. Information about whether a blank 101-1, . . . , 101-n is in the disk holder can be stored in an RFID tag on the disk holder.

In addition, it is no longer necessary to use a written material designation on the blank. This is helpful as the written material designation on the blank 101-1, . . . , 101-n is sometimes milled away. The legibility of the labeling on the blanks 101-1, . . . , 101-n is reduced by heavy use.

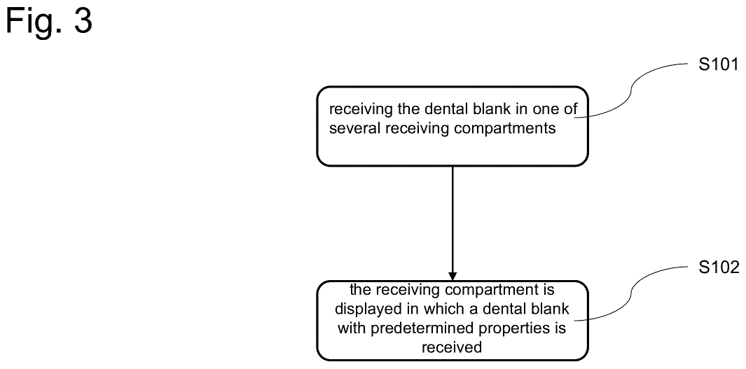

FIG. 3 shows a block diagram for managing dental blanks 101-1, . . . , 101-n. The method comprises the step S101 of receiving the dental blank 101-1, . . . , 101-n in one of several receiving compartments 103-1, . . . , 103-n. In step S102, the receiving compartment 103-1, . . . , 103-n is displayed in which a dental blank 101-1, . . . , 101-n with predetermined properties is received.

If several manufacturing machines 113 and disk managers 100 are used, they can also communicate with each other in color-coded form via the lighting. In addition, communication with a storehouse or storage cabinet can also be carried out. For example, a first manufacturing machine 113 is coded with the lighting color blue and a second manufacturing machine 113 with the lighting color green. In this way, a user can immediately recognize into which manufacturing machine 113 the respective blank 101-1, . . . , 101-n is to be inserted. However, the disk manager 100 can be permanently assigned to a specific manufacturing machine 113 so that it only receives orders from a specific manufacturing machine 113.

Communication with the storehouse takes place on a computer. By connecting the disk manager 100 to the manufacturing machine 113, the stored materials can be checked. The disk manager 100 can have its own display. The display of the manufacturing machine 113 or calling up a website via a mobile device can also be used to provide the user with information.

The disk manager 100 enables even an inexperienced user to recognize which blank is currently required for a manufacturing machine 113 and to load it into the manufacturing machine 113, since the respective receiving compartment 103-1, . . . , 103-n in the module 109 is lit up. The disk manager 100 thus offers user-friendly operation. New orders for blanks can be placed automatically or regularly by the disk manager 100, as it always checks the current stock level. For example, the disk manager 100 automatically orders new blanks if the stock falls below a specified minimum quantity.

A material forecast can also be made and statistics created to support orders. This ensures that there is always a minimum stock level in the storehouse. The statistics can also record which blank 101-1, . . . , 101-n is used the most. This allows the range to be reduced. A new blank 101-1, . . . , 101-n is only used if no used blank 101-1, . . . , 101-n is available. In addition, recommendations for stock management optimization can be issued.

In order to better understand the user's usage behavior with regard to blanks or material, it can be helpful to integrate the disk manager 100 into the workflow so that an optimal material flow can be achieved. The disk manager 100 can be used by a dentist or in a dental laboratory as a storage machine.

All the features explained and shown in connection with individual embodiments of the invention can be provided in different combinations in the subject matter according to the invention in order to realize their advantageous effects simultaneously.

All method steps can be implemented by devices that are suitable for executing the respective method step. All functions performed by the features of the subject matter can be a method step of a method.

The scope of protection of the present invention is given by the claims and is not limited by the features explained in the description or shown in the figures.

### REFERENCE LIST

100 Disk manager
101 Blanks
103 Receiving compartments
105 Display device
107 Storage device
109 Module
111 Input interface
113 Manufacturing machine
115 Scanning device

1. A disk manager for managing dental blanks, comprising:
several receiving compartments for receiving one dental blank each; and
a display device for displaying in which of the several receiving compartments a dental blank is received.

2. The disk manager according to claim 1,
wherein the disk manager comprises a storage device for storing the properties of the dental blank in the respective receiving compartment.

3. The disk manager according to claim 2,
wherein the properties comprise available remaining material, a color, an identification number, a size and/or a manufacturing material of the received blank.

4. The disk manager according to claim 1,
wherein the disk manager can be composed of several modules with at least one receiving compartment.

5. The disk manager according to claim 1,
wherein the disk manager comprises an input interface for inputting properties of a dental blank.

6. The disk manager according to claim 1,
wherein the receiving compartments comprise an inclination or a foam insert for stable positioning of the dental blank.

7. The disk manager according to claim 1,
wherein the disk manager comprises a scanning device for scanning a dental blank.

8. The disk manager according to claim 7,
wherein the scanning device comprises an optical camera, an RFID reader, a bar code reader and/or a QR code reader.

9. The disk manager according to claim 1,
wherein an antenna or a sensor for detecting the received dental blank is assigned to each receiving compartment.

10. The disk manager according to claim 1,
wherein a display device is assigned to each receiving compartment.

11. The disk manager according to claim 1,
wherein the display device comprises a light-emitting diode, a liquid crystal display or an electronic paper.

12. A module for a disk manager comprising at least one receiving compartment for receiving a dental blank.

13. The module according to claim 12,
wherein the module comprises a display device for displaying the receiving compartment.

14. The module according to claim 13,
wherein the module can be coupled horizontally or vertically.

15. A method for managing dental blanks, comprising
receiving a dental blank in one of several receiving compartments; and
displaying the receiving compartment in which a dental blank with predetermined properties is received.

16. The method according to claim 15, comprising
detecting whether a dental blank is inserted in one of the receiving compartments.

17. The method according to claim 15, comprising
detecting a remaining quantity of the dental blank.

18. The method according to claim 17,
wherein another dental blank is automatically reordered if the remaining quantity of the dental blank falls below a predetermined value.

19. The method according to claim 15,
wherein a stock of dental blanks is managed.
